(19) United States
(12) Patent Application Publication
(10) Pub. No.: US 2006/0140897 A1
(43) Pub. Date: Jun. 29, 2006

(54) Biphasic inducing agent for aqueous cleansing compositions

(75) Inventors: Rajesh Patel, Naugatuck, CT (US); Rosa Paredes, Shelton, CT (US); Daniel Pereira, Middletown, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 11/023,839

(22) Filed: Dec. 28, 2004

(51) Int. Cl. A61K 8/73

(52) U.S. Cl. 424/70.13

(57) ABSTRACT

The invention relates to the use of specifically defined agents that induce the formation of biphasic liquid cleansing compositions. In particular the biphasic inducing agents include water soluble, high-gel-point polysaccharides having a molecular weight greater than about 10,000 Daltons; fatty ester or fatty ether ethoxylates of intermediate ethoxylation; and mixtures thereof. Optional use of salt and polydextrose allows less total biphasic inducing agents to be used in the composition and more control over the physical properties of the composition.

### FIELD OF THE INVENTION

The present invention relates to aqueous liquid cleansing compositions that are biphasic in nature. More specifically, such compositions are characterized by having (assuming they have been standing a sufficiently long period of time after shaking) both an upper aqueous layer and a separate lower aqueous layer.

### BACKGROUND

Biphasic liquids defined by the general fact that the liquid is divided into two phases are not new. In some of these liquids one layer is an aqueous layer and the second layer is a water immiscible oily material while in others both layers are aqueous based.

U.S. Pat. No. 3,718,609 issued to Weimer on Feb. 27, 1973 discloses a liquid detergent composition having an aqueous layer and a layer of liquid water immiscible oily material. When shaken, the liquid forms a temporary oil-in-water emulsion.

Similarly, U.S. Pat. No. 3,810,478 issued to Olson Jr. et al on May 14, 1974 discloses a two-phase shampoo composition made by preparing substantially polar and lipophilic portions of a shampoo composition.

Biphasic compositions comprising an upper and lower aqueous phase are also disclosed in the art. U.S. Pat. No. 6,429,177 issued to Williams et. al. on Aug. 6, 2002 discloses biphasic compositions including 5 to 35% surfactant; 1 to 12% thickener; 4 to 20% polyalkylene glycol; and a sufficient amount of non-chelating mineral salt to induce phase separation.

U.S. Pat. No. 6,180,587 issued to Fuller et. al. on Jan. 30, 2001 disclose multiphase cleansing compositions having at least 1% of a polymer or copolymer selected from the group consisting of polyacrylate, polystyrene sulfonate, polyvinyl-pyrrolidone, maleic anhydride and their mixtures.

EP 0,116,422 to Harmer published on Apr. 6, 1988 also discloses multi-layered compositions in which two liquids are dispersible and which separate on standing. Sodium hexamataphosphate is a preferred biphasic inducing agent required in these compositions.

In U.S. Pat. No. 6,787,511 issued to Patel et al on Sep. 7, 2004, and U.S. Pat. No. 6,727,209 issued to Pereira et al on Apr. 27, 2004, the current inventors reported that polydextrose of molecular weight between about 600 and about 3, 000 Daltons, used alone or in combination with a salt such as MgSO₄ and/or sucrose, induced biphasic liquid formation. Although these polydextrose oligomers provided certain advantages such as not requiring salt and being highly cost-effective for the mass market, continuing work indicated that they had limitations. Firstly, the compositions tended to remain transparent after shaking so that a part of the novelty of the biphasic composition, which involves an optical transition from clear to opaque was not so pronounced.

Secondly, the polydextrose compositions, as well as the compositions described in U.S. Pat. No. 6,429,177 issued to Williams et. al. on Aug. 6, 2002 required a separate thickener to achieve a viscosity suitable for a liquid cleanser especially one targeted to personal cleansing, e.g., shower gel and shampoo.

The inventors have now discovered additional biphasic inducing agents (BIA) that can be used alone or in combination with polydextrose and/or salt. These new BIA fall into two classes. One class includes specific polysaccharides that surprisingly have higher molecular weights than the optimal polydextrose oligomers described above. The second class includes intermediate ethoxylates of specific fatty esters or fatty ethers.

The present invention seeks improvements over deficiencies in the known art. Among the one or more problems addressed include developing BIA that induce the formation of aqueous biphasic cleaning liquids that are economical, require no additional thickener and produce an optical transition from clear to opaque when shaken.

### BRIEF DESCRIPTION OF INVENTION

Unexpectedly, applicants have now found that biphasic liquids (e.g., liquids that separate into top and bottom aqueous liquids) may be induced by addition of sufficient quantity of specifically defined agents, designated “Biphasic Inducing Agents” (BIA). These BIA can be used alone or combined with and boost the efficiency of one or more know BIA, especially salt and polydextrose in inducing biphasic liquid formation.

More specifically, the liquid cleansing compositions include:
(a) about 5% to 75% by wt. of a surfactant selected from the group consisting of anionic surfactants, nonionic surfactants; amphoteric/zwitterionic surfactants, cationic surfactants and mixtures thereof;
(b) a Biphasic Inducing Agent (BIA) selected from the group consisting of a high-gel-point polysaccharides having a molecular weight greater than about 10,000 Daltons; an intermediate ethoxylate fatty ester or a fatty ether; and mixtures thereof;
(c) from 0 to about 10%, preferably from about 2% to about 10% and most preferably from about 2% to about 8% of a salt;
(d) from 0 to about 35%; preferably from about 5% to about 35% and most preferably from about 5% to about 15% of a polydextrose; and
   wherein the amount of Biphasic Inducing Agent is sufficient to produce at least two visibly separated aqueous based layers when the compositions are left undisturbed without stirring or shaking or other agitation.

### DETAILED DESCRIPTION OF INVENTION

As used herein % or wt % refers to percent by weight of an ingredient as compared to the total weight of the composition or component that is being discussed.

Except in the operating and comparative examples, or where otherwise explicitly indicated, all numbers in this description indicating amounts of material or conditions of reaction, physical properties of materials and/or use are to be understood as modified by the word “about.” All amounts are by weight of the final composition, unless otherwise specified.

It should be noted that in specifying any range of concentration, any particular upper concentration can be associated with any particular lower concentration.

For the avoidance of doubt the word “comprising” is intended to mean “including” but not necessarily “consisting of” or “composed of.” In other words, the listed steps or options need not be exhaustive.

The present invention relates to biphasic liquid cleansing compositions wherein the formation of the biphasic liquid is induced by the addition of sufficient amounts of particular Biphasic Inducing Agents (BIA). The general concept behind the invention is that, when sufficient amount(s) of BIA is (are) added, phase separation occurs. For example, this is shown in the Examples wherein, when 3% of a particular polysaccharide, pullulan (MW 200,000) is added, separation occurs. Different surfactant systems can be used and the specific type of surfactants is not a limiting factor although the type and level of surfactant can affect the amount of BIA required to induce phase separation in the composition.

The inventive compositions may be used in combination with a transparent package in order to view the liquid. Thus, in one embodiment, the invention is a system including a transparent or translucent package in combination with the liquid.

Typically, once the biphasic composition is formed (e.g., the composition “settles” after having been shaken), the viscosity of the lower layer is generally lower than that of the upper layer.

Also, the density of lower layer is typically greater than that of upper layer.

Typically, in such biphasic liquids, there is no recrystallization visible after composition has been standing for 6 months at room temperature.

The composition after being shaken should preferably have a shower-gel or shampoo like viscosity of 100 to 5000 mPas, more preferably 200 to 4000 mPas at a shear rate 10 s⁻¹ at 25° C. as measured with an appropriate viscometer such as a Haake RV20 Rotovisco Rheometer.

In a second embodiment, the BIAs of the invention are used with a small amount of salt or salt in combination with a polydextrose or a polyethylene glycol. In this case, it has been found the total level of all BIAs that is needed to induce biphasic liquid formation is reduced. In this embodiment, the composition typically includes about 1% to about 10% of a salt and about 2% to about 35% of a polydextrose in addition to the BIA of the invention.

In a third embodiment, about 2% to about 10% of a salt and about 5% to about 35% of a polydextrose is included in addition to the BIA of the invention.

The various components of the composition are discussed in greater detail below.

Surfactant

The surfactant generally will be included at a level from about 5% to about 75% by wt. of the total composition.

The surfactant may be selected from the group consisting of anionic surfactants, nonionic surfactants, amphoteric/zwitterionic surfactants, cationic surfactants and mixtures thereof. Preferably, there will be at least one anionic surfactant.

A. Anionic Surfactant

Non-limiting examples of anionic surfactants are disclosed in McCutcheon's Detergents and Emulsifiers, North American Edition (1986), published by Allured Publishing Corporation; McCutcheon's Functional materials, North Americas Edition (1992), both of which are incorporated by reference into the subject application.

Examples of anionic surfactants include sarcosinates, sulfates, isethionates, taurates, phosphates, lactylates, glutamates and mixtures thereof. Among isethionates are preferred alkoxyl isethionates such as sodium cocoyl isethionate, sodium lauroyl isethionate and mixtures.

The alkyl and alkyl ether sulfates typically have the respective formulae ROSO₃M and RO(C₂H₄O)ₓSO₃M, wherein R is alkyl or alkenyl of from about 10 to about 30 carbon atoms, x is from about 1 to about 10, and M is a water-soluble cation such as ammonium, sodium, potassium, magnesium and triethanolamine. Another suitable class of anionic surfactant are the water-soluble salts of the organic, sulfuric acid reaction products of the general formula:
R₁—SO₃-M
wherein R₁ is chosen from the group consisting of a straight or branched chain, saturated aliphatic hydrocarbon radical having from about 8 to about 24, preferably about 10 to about 16, carbon atoms; and M is a cation. Still other anionic synthetic surfactants include the class designated as succinates and sulfosuccinates, olefin sulfonates having about 12 to about 24 carbon atoms, and β-alkyloxy alkane sulfonates. Examples of these materials are sodium lauryl sulfate and ammonium lauryl sulfate.

Other anionic materials useful herein are soaps (i.e., alkali metal salts, e.g., sodium or potassium salts or ammonium or triethanolamine salts) of fatty acids, typically having from about 8 to about 24 carbon atoms, preferably from about 10 to about 20 carbon atoms. The fatty acids used in making the soaps can be obtained from natural sources such as, for instance, plant or animal-derived glycerides (e.g., palm oil, coconut oil, soybean oil, castor oil, tallow, lard, etc.). The fatty acids can also be synthetically prepared. Soaps are described in more detail in U.S. Pat. No. 4,557,853.

Other useful anionic materials include phosphates such as monoalkyl, dialkyl, and trialkylphosphate salts.

Other anionic materials include alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and alkanolamine (e.g., triethanolamine), a preferred examples of which are sodium lauroyl sarcosinate, sodium cocoyl sarcosinate, ammonium lauroyl sarcosinate, and sodium myristoyl sarcosinate. TEA salts of sarcosinates are also useful.

Also useful are taurates which are based on taurine, which is also known as 2-aminoethanesulfonic acid. Especially useful are taurates having carbon chains between C₈ and C₁₆. Examples of taurates include N-alkyltaurines such as the one prepared by reacting dodecylamine with sodium isethionate according to the teaching of U.S. Pat. No. 2,658,072 which is incorporated herein by reference in its entirety. Further non-limiting examples include ammonium, sodium, potassium and alkanolamine (e.g., triethanolamine) salts of lauroyl methyl taurate, myristoyl methyl taurate, and cocoyl methyl taurate.

Also useful are lactylates, especially those having carbon chains between C₈ and C₁₆. Non-limiting examples of lactylates include ammonium, sodium, potassium and alkanolamine (e.g., triethanolamine) salts of lauroyl lactylate, cocoyl lactylate, lauroyl lactylate, and caproyl lactylate.

Also useful herein as anionic surfactants are alkylamino carboxylates such as glutamates and glycinates, especially those having carbon chains between C₈ and C₁₆. Non-limiting examples include ammonium, sodium, potassium and alkanolamine (e.g., triethanolamine) salts of lauroyl glutamate, lauroyl glycinate, myristoyl glutamate and myristoy glycinate, and cocoyl glutamate and cocoyl glycinate.

Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate, and sodium lauroyl glycinate, cocoyl glycinate and mixtures thereof.

Especially preferred for use herein are ammonium lauryl sulfate, ammonium lauryl ether sulfate, sodium lauryl ether sulfate, sodium lauryl ether sulfosuccinates, sodium methyl 2-sulfolaurate, disodium 2-sulfolaurate, sodium lauroyl sarcosinate, sodium cocoyl sarcosinate, sodium myristoyl sarcosinate, sodium lauroyl lactate, and triethanolamine lauroyl lactylates.

B. Nonionic Lathering Surfactants

Non-limiting examples of nonionic lathering surfactants for use in the compositions of the present invention are disclosed in McCutcheon's, Detergents and Emulsifiers, North American Edition (1986), published by allured Published Corporation; and McCutcheon's, Functional materials, North American Edition (1992); both of which are incorporated by reference herein in their entirety.

Nonionic lathering surfactants useful herein include those selected form the group consisting of alkyl glucosides, alkyl polyglucosides, polyhydroxy fatty acid amides, alkoxylated fatty acid esters, alcohol ethoxylates, lathering sucrose esters, amine oxides, and mixtures thereof.

Alkyl glucosides and alkylipolyglucosides are useful herein, and can be broadly defined as condensation articles of long chain alcohols, e.g., C8-30 alcohols, with sugars or starches or sugar or starch polymers i.e., glycosides or polyglycosides. These compounds can be represented by the formula (S)ₙ—O—R wherein S is a sugar moiety such as glucose, fructose, mannose, and galactose; is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol and the like. Preferred examples of these surfactants include those wherein S is a glucose moiety, R is a C8-20 alkyl group, and n is an integer of from about 1 to about 9. Commercially available examples of these surfactants include decyl polyglucoside (available as APG 325 CS from Henkel) and lauryl polyglucoside (available as APG 600 CS and 625 CS from Henkel). Also useful are sucrose ester surfactants such as sucrose cocoate and sucrose laurate.

Other useful nonionic surfactants include polyhydroxy fatty acid amide surfactants, more specific examples of which include glucosamides, corresponding to the structural formula: 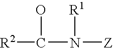
wherein R¹ is H, C₁-C₄ alkyl, 2-hydroxyethyl, 2-hydroxy-propyl, preferably C₁-C₄ alkyl, more preferably methyl or ethyl, most preferably methyl; R² is C₅-C₃₁ alkyl or alkenyl, preferably C₇-C₁₉ alkyl or alkenyl, more preferably C₉-C₁₇ alkyl or alkenyl, most preferably C₁₁-C₁₅ alkyl or alkenyl; and Z is a polyhydroxy hydrocarbyl moiety having a linear hydrocarbyl chain with at least 3 hydroxyl directly connected to the chain, or an alkoxylated derivative (preferably ethoxylated or propoxylated) thereof. Z preferably is a sugar moiety selected from the group consisting of glucose, fructose, maltose, lactose, galactose, mannose, xylose, and mixtures thereof. As especially preferred surfactant corresponding to the above structure is coconut alkyl N-methyl glucoside amide (i.e., wherein the R²CO-moiety is derived form coconut oil fatty acids). Processes for making compositions containing polyhydroxy fatty acid amides are disclosed, for example, in GB Patent Specification 809,060, published Feb. 18, 1959, by Thomas Hedley & Co., Ltd.; U.S. Pat. No. 2,965,576, to E. R. Wilson, issued Dec. 20, 1960; U.S. Pat. No. 2,703,798 to A. M. Schwartz, issued Mar. 8, 1955; and U.S. Pat. No. 1,985,424, to Piggott, issued Dec. 25, 1934; which are incorporated herein by reference in their entirety.

Other examples of nonionic surfactants include amine oxides. Amine oxides correspond to the general formula R₁R₂R₃N→O, wherein R₁ contains an alkyl, alkenyl or monohydroxyl alkyl radical of from about 8 to about 18 carbon atoms, and R₂ and R₃ contain from about 1 to about 3 carbon atoms and from 0 to about 1 hydroxy group, e.g., methyl, ethyl, propyl, hydroxyethyl, or hydroxypropyl radicals. The arrow in the formula is a conventional representation of a semipolar bond. Examples of amine oxides suitable for use in this invention include dimethyldodecylamine oxide, oleyl di(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, diemethylhexadecyclamine oxide.

Another useful class of nonionic surfactant is the foam boosting amides. Although these materials do not lather by themselves they often boost lather when combined with other surfactants such as alkyl ethoxy sulfates. Examples of suitable amides include coco monoethanol amide, and ethoxyleted coco monoethanol amide, e.g., PEG-5 CMEA.

Non-limiting examples of preferred nonionic surfactants for use herein are those selected form the group consisting of C₈-C₁₄ glucose amides, C₈-C₁₄ alkyl polyglucosides, sucrose cocoate, sucrose laurate, lauramine oxide, cocoamine oxide, coco monothanolamide PEG-5 coco monoethanolamide and mixtures thereof.

C. Amphoteric Lathering Surfactants

The term “amphoteric lathering surfactant,” as used herein, is also intended to encompass zwitterionic surfactants, which are well known to formulators skilled in the art as a subset of amphoteric surfactants.

A wide variety of amphoteric lathering surfactants can be used in the compositions of the present invention. Particularly useful are those which are broadly described as derivatives of aliphatic secondary and tertiary amines. Here preferably the nitrogen is in a cationic state, the aliphatic radicals can be straight or branched chain and one of the radicals contains an ionizable water solubilizing group, e.g., carboxy, sulfonate, sulfate, phosphate, or phosphonate.

Non-limiting examples of amphoteric surfactants useful in the compositions of the present invention are disclosed in McCutcheon's, Detergents and Emulsifiers, North American Edition (1986), published by Allured Publishing Corporation; and McCutcheon's, Functional Materials, North American Edition (1992); both of which are incorporated by reference herein in their entirety.

Non-limiting examples of amphoteric or zwitterionic surfactants are those selected from the group consisting of betaines, sultaines, hydroxysultaines, alkyliminoacetates, iminodialkanoates, aminoalkanoates, and mixtures thereof.

Examples of betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (available as Lonaine 16SP from Lonza Corp.), lauryl bis-(2-hydroxyethyl) carboxymethyl betaine, oleyl dimethyl gamma-carboxypropyl betaine, lauryl bis-(hydroxypropyl)alpha-carboxyethyl betaine, coco dimethyl sulfopropyl betaine, lauryl dimethyl sulfoethyl betaine, lauryl bis-(2-hydroxyethyl) sulfopropyl betaine, amidobetaines and amidosulfobetaines (wherein the RCONH(CH₂)₃ radical is attached to the nitrogen atom of the betaine), oleyl betaine (available as amphoteric Velvetex OLB-50 from Henkel), and cocamidopropyl betaine (available as Velvetex BK-35 and BA-35 from Henkel).

Example of sultaines and hydroxysultaines include materials such as cocamidopropyl hydroxysultaine (available as Mirataine CBS from Rhodia).

Preferred for use herein are amphoteric surfactants having the following structure: 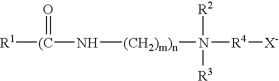
wherein R¹ is unsubstituted, saturated or unsaturated, straight or branched chain alkyl having from about 9 to about 22 carbon atoms. Preferred R¹ has from about 11 to about 18 carbon atoms; m is an integer from 1 to about 3, more preferably from about 2 to about 3, and more preferably about 3; n is either 0 or 1, preferably 1; R² and R³ are independently selected from the group consisting of alkyl having from 1 to about 3 carbon atoms, unsubstituted or mono-substituted with hydroxy, preferred R² and R³ are CH₃; X is selected form the group consisting of CO₂, SO₃ and SO₄; R⁴ is selected form the group consisting of saturated or unsaturated, straight or branched chain alkyl, unsubstituted or mono-substituted with hydroxy, having from 1 to about 5 carbon atoms. When X is CO₂, R⁴ preferably has 1 to 3 carbon atoms, more preferably 1 carbon atom. When X is SO₃ or SO₄, R⁴ preferably has from about 2 to about 4 carbon atoms, more preferably 3 carbon atoms.

Examples of amphoteric surfactants of the present invention include the following compounds:

Cetyl dimethyl betaine (this material also has the CTFA designation cetyl betaine);

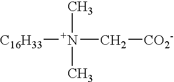

Cocamidopropylbetaine

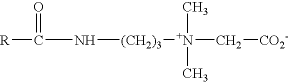

wherein R has from about 9 to about 13 carbon atoms

Cocamidopropyl hydroxy sultaine

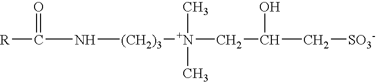

wherein R has from about 9 to about 13 carbon atoms.

D. Cationic Surfactants

Cationic surfactants are another useful class of surfactants that can be employed as auxiliary agents. They are particularly useful as additives to enhance skin feel, and provide skin conditioning benefits. One class of cationic surfactants is heterocyclic ammonium salts such as cetyl or stearyl pyridinium chloride, alkyl amidoethyl pyrrylinodium methyl sulfate, lapyrium chloride.

Tetra alkyl ammonium salts is another useful class of cationic surfactants. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl) dimethyl ammonium halides; behenyl dimethy ammonium chloride.

Other types of cationic surfactants that can be employed are the various ethoxylated quaternary amines and ester quats. Examples are PEG-5 stearyl ammonium lactate (e.g., Genamin KSL manufactured by Clarion), PEG-2 coco ammonium chloride, PEG-15 hydrogenated tallow ammonium chloride, PEG 15 stearyl ammonium chloride, dialmitoyl ethyl methyl ammonium chloride, dipalmitoyl hydroxyethyl methyl sulfate, strearyl amidopropyl dimethylamine lactate.

Still other useful cationic surfactants are quaternized hydrolysates of silk, wheat, and keratin proteins.

Biphasic Inducing Agent

BIA falling into one of two broad classes of water-soluble materials have been found useful in the practice of the present invention: high-gel-point polysaccharides and intermediate ethoxyltes of fatty esters or fatty ethers.

A. High-Gel-Point Polysaccharides

The polysaccharides of the invention have a molecular weight of at least about 10,000, preferably greater than 50,000, and most preferably greater than 150,000 Daltons. These polysaccharides are also water soluble, preferably highly water soluble (e.g., dissolve at a level of at least about 10% by weight in water) and most preferably form stable solutions at ambient temperature. The most preferred polysaccharides also form clear isotropic solutions in water at room temperature.

It has been found that in addition to the general properties described above, the polysaccharide should have a high gel point to function optimally as a BIA of the invention. This high gel point is indicative of a low tendency to form gels in water in the sense described below. By the term “gel” is meant a viscoelastic liquid or paste exhibiting an apparent yield stress. By the term “gel point” is meant the concentration in water where the polysaccharide forms a gel at room temperature (about 25° C.). By the term “high-gel-point polysaccharide” is meant a polysaccharides that either will not gel at any concentration or will induce biphasic formation when incorporated into the composition of interest at a lower concentration than is required for the polysaccharide to form a gel in water. This latter condition is generally achieved if the instant polysaccharide BIA have a gel point in water (minimum concentration to produce a viscoelastic liquid exhibiting a yield value) of at least about 2% by weight, more preferably of at least about 5% in water, and most preferably of at least about 10% in water. A test to determine the concentration of BIA required to induce phase separation is described in the EVALUATION METHODOLOGY SECTION below.

Without wishing to be bound by theory, suitable polysaccharides tend to be nonionic in character, and tend to have a molecular structure that tends to retard molecular association in water, e.g., branching, kinks or small pendant groups appearing with sufficiently high frequency, i.e., short repeat lengths.

Many such polysaccharides are described in the treatise edited by R. L. Whistler and J. N. BeMiller, “Industrial Gums: Polysaccharides and Their Derivatives”, 3^{rd} Edition, Academic Press Inc., San Diego, Calif. (1993), herein after called “Whistler” and incorporated by reference herein in its entirety.

One suitable class of non-gelling polymers is pullulan which is described in Whistler, Chapter 16, p 447. Pullulan is a class of glucans elaborated extracellularly by the fungus Aureobasidium (also known as Pullularia). Pullulan dissolves readily in water to form stable viscous solutions that do not gel.

The structure of Pullan is reported to be predominantly based on maltotriose units joined by α-D-(1-6) linkages.

Depending upon the strain of fungus employed, the molecular weight of pullulan an can vary between 1,000 to 3,000,000 Daltons. However, for the purposes of the present application, the molecular weight should be greater than about 10,000, preferably greater than 50,000 and most preferably between 100,000 and 300,00 Daltons. Pullulan is available from Hayashibara and sold under the trade name Pullulan PI 20. A particular pullulan from Hayashibara having an average molecular weight of 200,000 was suitable.

A second useful polysaccharide is one derived from high amylopectin starch. Amylopectin is one of the primary polysaccharide polymer components of starch, the other polysaccharide polymer component being amylose.

Amylopectin molecules have a branch-on-branch structure and are composed of chains of α-D-(1-4) linked glucopyranose of various lengths joined via unequally spaced α-D-(1-6) linkages. The molecular weight of amylopectin as it occurs in starch is purported to be in the range from about 5×10⁷ to about 4×10⁸ Daltons.

Amylopectin, in contrast with amylose, forms clear stable solutions (e.g., little retrogradation) when heated to a sufficiently high temperature and either does not form gels or only forms weak gels).

High amylopectin starches are available commercially as waxy variants (mutants) of corn, wheat and rice starch among others. Amylopectin-rich starches can also be modified to reduce molecular weight (e.g., heating solutions under high shear or partial hydrolysis with acid), or to make them more cold water soluble (e.g., gelatinized starch). Examples of such modified waxy starch include depolymerized or “thinned” waxy corn and pregelatinized waxy corn. Both modified waxy starches can be obtained for example, from National Starch and Chemicals.

Another class of polysaccharides is depolymerized or thinned starch. These materials differ from polydextroses in the degree of depolymerization—see below). The thinned starches generally have an average degree of polymerization of greater than 60, which corresponds to a molecular weight greater than about 10,000 Daltons.

Examples of depolymerized starches include: acid-modified starch which are generally formed by treating native or modified starch granules with dilute mineral acid at a temperature below that required for gelatinization; enzyme-converted starch which are starches treated with alph-amylase (or isoamylase to reduce branching) to achieve a lower viscosity, clearer aqueous mixture; oxidized starch treated with for example, ammonium persulfate; and thermomechanical degraded starches—slurry heated to high temperature under high shear.

Still another useful class of polysaccharide is exudate gums. An example of a useful exudate gum is gum arabic, which is a gum exuded from the bark of the Acacia tree. Gum arabic, is readily soluble in cold water, does not form gels, and forms clear solutions which have a relatively low viscosity.

One especially suitable high-gel-point polysaccharide is pullulan.

B. Fatty Ester or Fatty Ether Intermediate Ethoxylates

A second class of useful BIA is the intermediate ethoxylates of fatty esters or fatty ethers. By the term “intermediate ethoxylates” is meant ethoxylates that are longer than typical alcohol ethoxylate nonionic surfactants (greater than about 12, preferably greater than about 15), but lower than hydrophobically modified polyoxyethylene copolymers that are used as thickeners (lower than about 100, preferably lower than about 70). The degree of ethoxylation (defined as moles ethylene oxide per molecule) of the intermediate fatty ethoxylate is in the range from about 10 to about 80, preferably about 15 to about 45 and most preferably from about 15 to 35 moles ethylene oxide per molecule.

By the term “fatty ester” or “fatty ether” is meant esters or ethers that incorporate “fatty chains” by which is generally meant one or more straight or branched alkyl or alkenyl chains having from about 8 to about 22 carbon atoms.

Suitable fatty ester intermediate ethoxylates include the C₁₂-C₂₂ saturated or unsaturated fatty acid mono and diglyceride ethoxylates with an average of 20 to 80 units or moles ethylene oxide per molecule. Examples include ethoxylated castor oil, ethoxylated soya oil glycerides, and ethoxylated olive oil glycerides, and ethoxylated palm kernal glycerides. Examples include PEG-30 castor oil, PEG-40 hydrogenated castor oil, PEG 36-soya glycerides, PEG-75 Soya ethoxylate, PEG-40 olive oil glycerides, and PEG-47 palm kernal glycerides.

Another fatty ester intermediate ethoxylate includes the C₁₂-C₂₂ saturated or unsaturated fatty acid sorbitan esters having about 20 to about 80 moles of ethylene oxide per molecule. Examples include PEG-40 sorbitan laurate, PEG-75 sorbitan monolaurate, PEG-40 sorbitan trioleate, and PEG-50 sorbitan laurate.

Another fatty ester intermediate ethoxylate is a C₁₂-C₂₂ fatty acid ethoxylated with an average of about 20 to about 50 ethylene oxide units. An example PEG-20 myristate.

Fatty ether intermediate ethoxylates include C₁₂-C₂₂ saturated or unsaturated (preferably saturated) fatty alcohols ethoxylated with an average of about 12 to about 80 ethylene oxide units. Examples include steareth-25, steareth-50, laureth-40, and beheneth-30.

Castor oil ethoxylate having 20 to 50 moles ethylene oxide is a particularly suitable fatty ester intermediate ethoxylate.

The level of BIA required depends on the cleanser composition and upon the ratio of the separate aqueous phases that is desired. Thus, for example, if the composition contains salt, or a polydextrose of molecular weight less than 3600 Daltons, as little as 0.5% of a non-gelling polysaccharide of the invention may be required by weight of composition while 2-10%, preferably 2-5%, may be required in the absence of the salt and polydextrose. Furthermore, when the non-gelling polysaccharide and fatty intermediate ethoxylate are combined, the levels of each separate BIA required will vary according to the combination.

The level of high-gel-point polysaccharide required will generally be about 0.25 to about 20%, preferably 0.25 to about 10% and most preferably about 0.5% to about 5% based on the total weight of the composition, i.e., all phases taken together.

The level of fatty intermediate ethoxylate required will generally be about 0.5 to about 15%, preferably 0.5 to about 10% and most preferably about 0.1% to about 5% based on the total weight of the composition, i.e., all phases taken together.

There is also generally a balance between amount of surfactant used and amount of BIA. Generally lower surfactant requires more BIA and, conversely, more surfactant requires less BIA. Thus, for example, 5% to 10% by wt. surfactant may require about 5-10% high-gel-point polysaccharide while 10%-20% surfactant may need only about 1-5% of the same low-gelling polysaccharide.

Optional Ingredients

Polydextrose and Salt as Optional Biphasic Inducing Agents

Both polydextrose and salts are useful as optional biphasic inducing agent used in combination with the BIA of the present invention. Polydextrose is a relatively low molecular weight, generally branched polymer primarily composed of D-glucose:

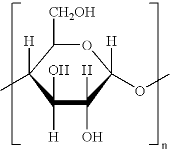

wherein n (defining number of linking glucose units) is from about 4 to about 22.

As defined herein, the term polydextrose includes maltodextrin, which is formed by the extensive depolymerization of starch through, for example, dry-roasting, and synthetic poydextrose which is formed by heating dextrose (glucose) with an acid catalyst.

Polydextrose, having a MW in the range of from 600 to about 3600, more preferably 700 to 3000, more preferably 700 to 1800, and even more preferably 900 to 1500 is known to induce the formation of biphasic liquids on its own. These polymers are also highly suitable for use in combination with the BIA of the present invention and can be used to advantage to improve the overall composition and its economics.

Electrolytes preferably, non-chelating electrolyte (chelating electrolytes have generally poorer biodegradability), are also useful in combination with the instant BIA.

Typically, the electrolyte should be a salt of a sulphate, bisulfate, carbonate, bicarbonate, phosphate, chloride, etc. Examples include sodium sulphate, potassium sulphate, ammonium sulphate, sodium chloride, and magnesium chloride. Magnesium sulphate and sodium chloride are particularly preferred.

Other Optional Ingredients

The following optional ingredients may be used in the multiphasic/biphasic compositions of the invention.

The composition may contain polyalkylene glycol. The polyalkylene glycol should be an alcohol, glycol or polyether of minimal molecular weight which is not irritating to the skin.

Examples of such include alcohols, particularly polyalkylene oxides having MW 200-6000, preferably 200 to 3000. The polyalkylene glycol can be comprised of ethylene oxide, propylene oxide, butylene oxide or their mixtures either as polymers or copolymers. Specific examples include polyethylene glycols such as PEG 400. As noted, use of such alcohols is not required.

The composition may further comprise thickeners. Generally, the thickener/viscosity modifier serves to thicken the upper and/or lower layer.

Thickeners which may be used include hydrophobically modified polyethers. Examples of this class of thickeners which may be used include but are not limited to sugar esters such as PEG (160) sorbitan triisostearate (Rheodol TWS-399C ex Kao Chemicals) or PEG-120 Pentaerythrityl Tetrastearate ex Croda. Other examples include Glucam DOE 120 (PEG 120 Methyl Glucose Dioleate); Rewoderm® (PEG modified glyceryl cocoate, palmate or tallowate) from Rewo Chemicals; Antil® 141 (from Goldschmidt); and Carbopol® polymers from Noveon.

Another class of suitable polymers is hydrophobically modified cellulose ethers including but not limited to hydroxyethyl cellulose, hydroxypropylcellulose and cellulose ethers with long pendant chains such as nonoxynyl hydroxyethylcellulose (Amerchol Polymer HM 1500).

Another class of suitable polymers is the hydrophobically modified acrylate copolymers such as Antil 208® (ex Goldschmidt) (acrylate/steareth-50 acrylate copolymer).

Another class of suitable polymers is the hydrophobically modified polyurethanes such as Acrysol series (e.g., Acrysol RM-2020) from Rhom and Haas.

Another class of suitable thickeners is gums such as xanthan gums, guar gums and chemically modified guar gums.

In addition to the ingredients noted above, the compositions of the invention may contain hydrotropes including but not limited to short chain monohydric or dihydric alcohols, xylene sulphonate and hexylene glycol whose purpose is to avoid the formation of liquid crystal phases resulting from the separation of the surfactant material into the upper phase hence increasing its apparent concentration.

The compositions may comprise benefit agents. Benefit agent may be any material that has potential to provide an effect on, for example, the skin and/or the hair.

The benefit agent may be water insoluble material that can protect, moisturize or condition skin or hair upon deposition from compositions of invention. These may include silicon oils and gums, fats and oils, waxes, hydrocarbons (e.g., petrolatum), higher fatty acids and esters, vitamins, sunscreens. They may include any of the agents, for example, mentioned at column 8, line 31 to column 9, line 13 of U.S. Pat. No. 5,759,969, hereby incorporated by reference into the subject application.

The benefit agent may also be a water soluble material such as glycerin, polyols (e.g., saccharides), enzyme and α- or β-hydroxy acid either alone or entrapped in an oily benefit agent.

The benefit agent may be found in either the upper or the lower layer depending on its solubility and partition coefficient, for example, oil may partition into the upper layer while more water soluble agents (e.g., α-hydroxyacids) may go into the lower.

The compositions may comprise perfumes, sequestering agents such as EDTA, EHDP in amounts 0.01 to 1%, preferably 0.01 to 0.05%; coloring agents, opacifiers and pearlizers such as zinc stearate, magnesium stearate, TiO₂, mica, EGMS (ethylene glycol monostrearate) or styrene/acrylate copolymers.

The compositions may further comprise antimicrobials such as 2-hydroxy 4,2′4′ trichlorodiphenylether (DP300), 3,4,4′-trichlorocarbanilide, essential oils and preservatives such as dimethyl hydantoin (Glydant XL 1000), anti-dandruff agents, parabens, sorbic acid etc.

The compositions may also comprise coconut acyl mono or diethanol amides as suds boosters, and strongly ionizing salts such as sodium chloride and sodium sulfate may also be used to advantage.

Antioxidants such as, for example, butylated hydroxytoluene (BHT) may be used advantageously in amounts of about 0.01% or higher if appropriate.

Cationic conditioners which may be used including Quatrisoft LM-200 Polyquaternium-24, Merquat Plus 3330-Polyquaternium 39; and Jaguar® type conditioners.

Composition may also include clays such as Bentonite® claims as well as particulates such as abrasives, glitter, and shimmer agents (e.g., micas).

The balance of the composition is generally water.

The compositions of the invention, when unmixed, have a viscosity of the lower layer which is generally lower than the viscosity of the upper layer and a density of the lower layer which is greater than the density of the upper layer.

The compositions of the invention, in a separated state, are also stable in that no recrystallization (e.g., in the lower layer) occurs even when left sitting for more than 6 months at temperature of 0° C.

Compositions of the invention have an experiential element in that they are intended to be agitated by the consumer to mix and form a single visible phase before separating again after a time, anywhere from about a few seconds to not more than about 24 hours.

Finally, the packages in which the compositions are contained is preferably translucent or transparent. By this is meant that the materials (e.g., plastics) have a light transmittance of greater than 50%, preferably greater than 75%, more preferably greater than 85% as measured at wavelength of 460 nm as determined by standard spectroscopy method. In practical terms the package should be sufficiently transparent to permit the separation of the two or more layers to be visible to the naked eye.

Evaluation Methodology:

A. Measurement of Viscosity

Viscosity was measured with a Haake model RV 20 Rotovisco rheometer which includes a stand and sample temperature control unit, cups and bobs for loading the sample, a water bath which is maintained at 25° C. and a computer and plotter to manipulate and record the data. A NV cup/bob assembly is employed for viscosity measurements of low viscous products, e.g. diluted solutions, fruit juices, etc. An SV1 cup/bob assembly is employed for viscosity measurements of intermediate viscosity liquids, e.g., typical shower gel or shampoo products;

The viscosity was measured as follows. The rotor (bob) was secured to the top segment of the viscometer and the RV 20 viscometer was adjusted using the zero button. A test sample was poured into the cup until almost three fourths filled (approx. 20 g). If the test sample is a biphasic liquid it was vigorously stirred but with minimum air bubble entrapment. In a control experiment the liquid was first allowed to sit undisturbed for the same amount of time as would be required to complete a viscosity measurement. If no detectable separation was observed during this period, a fresh biphasic sample was vigorously stirred as above and loaded into the viscometer cup. The cup was carefully slid through the temperature controller and screwed to the main segment of the rheometer so that the rotor was immersed in the product and sample was slightly above the rim of the rotor. After 5 to 10 minutes equilibration, a programmed stress-strain sweep was initiated. Generally four shear-rate-segments consisting of 10 steps each were selected. The shear rates were 1, 10, 100, 400 sec⁻¹.

The viscosity was computed from the measured rheogram and expressed as mPas (cps) at each of the four shear rates.

B. Evaluation of Biphasic Formation

A surfactant solution is prepared at about 5 wt. % to about 35.0 wt. % that contained any additional desired ingredients. The composition is allowed to equilibrate undisturbed overnight to confirm that no phase separation occurrs, i.e., a stable single-phase composition and not a biphasic liquid is formed. The composition is then divided into two parts. To one part is added an appropriate amount of a candidate BIA, e.g., pullulan, to produce a desired concentration, e.g., 3 wt % (generally but not always the candidate BIA is added via a solution). This resulting composition is termed the “Test Composition”. The other part serves as a “Reference Composition” once the same weight of water is added as the combined weight of water plus candidate BIA that is added to make the “Test Composition”.

The Test and Reference compositions each in sealed containers are then stirred for 1 hour at 60° C. until homogeneous and then are allowed to equilibrate overnight. The samples are then evaluated for phase separation. To qualify as a Biphasic Inducing Agent, a Test Composition must exhibit at least two aqueous phases while its corresponding Reference Composition exhibits only one aqueous phase.

The above experiment can then be repeated any number of times at a lower or higher BIA concentration in the test composition. The lowest BIA concentration that just induces visible phase separation is termed the “Minimum Biphasic Induction Concentration” (MBIC). In practice, a series of solution pairs are prepared and tested at the same time to determine the MBIC. The MBIC of the polysaccharide Biphasic Inducing Agent of the invention should be lower than its gel point in water.

C. Viscosity and Product Appearance

Compositions are evaluated for viscosity using the method described above. The compositions are also observed for any discoloration and re-crystallisation of saccharides at room temperature. Finally, relative opacity of a biphasic composition contained in a standard clear glass container is determined by eye just after it is shaken.

### EXAMPLES

### Example 1 and Comparative 1-3

Pullulan was examined for its ability to promote the formation of aqueous biphasic cleansers in comparison with a reference composition, and two compositions that employ prior art Biphasic Inducing Agents. These compositions were prepared by the methods described above in the EVALUATION METHODOLOGY section. The compositions and results are set forth in Table 1 below.

The results in Table 1 demonstrate that pullulan induces biphasic formation compared with its corresponding single aqueous phase Reference Composition (C 1), i.e., when the high-gel-point polysaccharide is substituted for water in C 1. The results demonstrate the advantages of pullulan in three key areas. Firstly, a significantly lower total amount of biphasic inducing agents is required in the case of pullulan (6%—Ex 1) relative to compositions employing only prior art biphasic inducing agents (23%—C 3, and 19%—C 4).

Secondly, the pullulan composition does not require an additional thickener to achieve a similar viscosity of the shaken biphasic liquid. In fact, Ex 1 has numerically the highest viscosity of the biphasics shown even though it contains the lowest total amount of BIA and no additional thickener.

Finally, the pullan biphasic, Ex 1, is opaque after shaking in contrast to the clear biphasic dispersion using the lower molecular weight prior art polydextrose, Maltrin M180.

TABLE 1
Compositions and results for Example 1 and Comparatives 1-3

| | | C1 | | |
|---|---|---|---|---|
| INGREDIENTS | Ex 1 | Ref for Ex 1 | C2 | C3 |
| SURFACTANT | Wt % | | | |
| Sodium Laureth Sulfate | | | | 16 |
| Cocoamidopropylbetaine | | | | 3 |
| Ammonium Laureth sulfate (1/2 EO) | 4.6 | 4.6 | 4.6 | |
| Ammonium Lauryl sulfate | 6.1 | 6.1 | 6.1 | |
| Cocomonoethanolamide | 1.0 | 1.0 | 1.0 | |
| PEG-5 cocomonoethanolamide | 0.5 | 0.5 | 0.5 | |
| Alpha-step BSS-45^{a} | 2 | 2 | 2 | |
| BIPHASIC INDUCING AGENT OF | | | | |
| INSTANT INVENTION | | | | |
| Pullulan | 3 | | | |
| BIPHASIC INDUCING AGENTS OF | | | | |
| PRIOR ART | | | | |
| MgSO₄ | 3 | 3 | 3 | 8 |
| Maltrin M180 | | | 20 | |
| PEG 800 | | | | 11 |
| ADJUNCT INGREDIENTS | | | | |
| Glycerox 767 | | | 1 | |
| Glycerin | | | 0.5 | |
| Rewoderm LIS80 | | | | 4 |
| Polyox 300N | | | 0.25 | |
| Perfume, colorant, preservatives | 1.5 | 1.5 | 1.5 | 1.5 |
| WATER | To 100% | To 100% | To 100% | To 100% |
| Number of Aqueous Phases | 2 | 1 | 2 | 2 |
| after sitting w/o stirring/shaking | | | | |
| Appearance of Shaken Sample | Opaque | Transparent | Transparent | Opaque |
| Viscosity Shaken Sample, | 1252 | — | 799 | 1227 |
| cps @25° C. | | | | |

### Example 2-4 and Comparative 4

Examples 2-4 illustrate the ability of a fatty ester of intermediate ethoxylation to induce the formation of biphasic liquid cleanser when sustituted for water in a single aqueous phase Reference Composition, C4. The compositions were prepared by the methods described above. The compositions and results are set forth in Table 2 below.

The results in Table 2 demonstrate that the ethoxylated polyol fatty ester, in this case an ethoxylated castor oil, PEG-30 castor oil indeed induces the formation of a biphasic liquid. As the level of PEG-30 caster oil increases the relative proportions of upper and lower phase change which alters the overall appearance of the composition.

TABLE 2
Compositions and results for Examples 2-4 and Comparative 4

| | | | | C4 |
|---|---|---|---|---|
| INGREDIENTS | Ex 2 | Ex 3 | Ex 4 | Ref for Ex 2-Ex 4 |
| SURFACTANT | Wt % | | | |
| Ammonium Laureth sulfate (1/2 EO) | 4.6 | 4.6 | 4.6 | 4.6 |
| Ammonium Lauryl sulfate | 6.1 | 6.1 | 6.1 | 6.1 |
| Cocomonoethanolamide | 1.0 | 1.0 | 1.0 | 1.0 |
| PEG-5 cocomonoethanolamide | 0.5 | 0.5 | 0.5 | 0.5 |
| Alpha-step BSS-45^{a} | 2 | 2 | 2 | 2 |
| BIPHASIC INDUCING AGENT OF | | | | |
| INSTANT INVENTION | | | | |
| PEG-30 Castor Oil | 2 | 4 | 6 | |
| BIPHASIC INDUCING AGENTS OF | | | | |
| PRIOR ART | | | | |
| MgSO₄ | 3 | 3 | 3 | 3 |
| Maltrin M180 | 15 | 15 | 15 | 15 |
| ADJUNCT INGREDIENTS | | | | |
| Perfume, colorant, preservatives | 1.5 | 1.5 | 1.5 | 1.5 |
| WATER | To 100% | To 100% | To 100% | To 100% |
| Number of Aqueous Phases | 2 | 2 | 2 | 1 |
| after sitting w/o stirring/shaking | | | | |
| Top Layer, cm | 0.9 | 1.8 | 2.4 | |
| Bottom Layer, cm | 6.6 | 5.7 | 5.0 | |
| Appearance of Shaken Sample | Turbid | Turbid | Turbid | N/A |

### Example 5-6 and Comparative 5-6

Example 5 and 6 illustrate the use of Pullulan in combination with other biphasic inducing agents. The compositions Ex 5, Ex 6, C5 and C6 were prepared by the methods described above. The compositions and results are set forth in Table 3.

The results in Table 3 demonstrate that the inclusion of relatively low amounts of pullulan, e.g., 0.5% in the case of Ex 2, in a homogenous one-phase liquid can induce the formation of a biphasic liquid compared to at least 5% additional Maltrin which would be required in the absence of the pullulan.

The exact level of pullulan required depends on the specific composition, in particular the level and types of total surfactant as well as additional BIA.

TABLE 3
Compositions and results for Examples 5-6 and Comparatives 5—5

| | | | C5 | C6 |
|---|---|---|---|---|
| INGREDIENTS | Ex 5 | E 6 | Ref for Ex 5 | Ref for Ex 6 |
| SURFACTANT | Wt % | | | |
| Ammonium Laureth sulfate (1/2 EO) | 4.6 | 4.6 | 4.6 | 4.6 |
| Ammonium Lauryl sulfate | 6.1 | 6.1 | 6.1 | 6.1 |
| Cocomonoethanolamide | 1.0 | 1.0 | 1.0 | 1.0 |
| PEG-5 cocomonoethanolamide | 0.5 | 0.5 | 0.5 | 0.5 |
| Alpha-step BSS-45^{a} | 2 | 2 | 2 | 2 |
| MgSO₄ | 4 | 3 | 3 | 3 |
| Maltrin M180 | 15 | 15 | 15 | 15 |
| BIPHASIC INDUCING AGENTS | | | | |
| OF INSTANT INVENTION | | | | |
| Pullulan | 0.5 | 1.0 | — | — |
| PEG 30 Castor Oil | 1 | | 1 | |
| BIPHASIC INDUCING AGENTS | | | | |
| OF PRIOR ART | | | | |
| ADJUNCT INGREDIENTS | | | | |
| Perfume, colorant, preservatives | 1.5 | 1.5 | 1.5 | 1.5 |
| WATER | To 100% | To 100% | To 100% | To 100% |
| Number of Aqueous Phases | 2 | 2 | 1 | 1 |
| after sitting w/o stirring/shaking | | | | |
| Ratio of thickness of Top Layer to | 70/30 | 70/30 | | |
| thickness of Bottom Layer | | | | |
| Appearance of Shaken Sample | Opaque | Opaque | Transparent | Transparent |
| Viscosity Shaken Sample, | 638 | 1007 | | |
| cps @25° C. | | | | |

### Example 7-8 and Comparative 8

Examples 7-8 illustrates the ability of other high gel point polysaccharides to induce the formation of biphasic liquid cleanser when incorporated into a single aqueous phase reference composition, C7. The compositions were prepared by the methods described above. The compositions and results are set forth in Table 4 below. N-Oil® is a tapioca dextrin, N-Lite® is a dextrin derived from waxy maize, and HI MAIZE 260 is an acid thinned corn starch, all available from National Starch and Chemicals. All these high-gel-point polysaccharides have an average molecular weight greater than 10,000 Daltons.

The results in Table 4 demonstrate that the thinned or depolymerized high-gel-point starches, in this case acid thinned, indeed induces the formation of a biphasic liquid.

TABLE 4
Compositions and results for Examples 7-8 and Comparatives 7

| | | | C7 |
|---|---|---|---|
| | | | Ref for |
| INGREDIENTS | Ex 7 | Ex 8 | Ex 7-Ex 8 |
| SURFACTANT | Wt % | | |
| Ammonium Laureth sulfate (1/2 EO) | 4.6 | 4.6 | 4.6 |
| Ammonium Lauryl sulfate | 6.1 | 6.1 | 6.1 |
| Cocomonoethanolamide | 1.0 | 1.0 | 1.0 |
| PEG-5 cocomonoethanolamide | 0.5 | 0.5 | 0.5 |
| Alpha-step BSS-45^{a} | 2 | 2 | 2 |
| MgSO₄ | 3 | 5% | 3 |
| Maltrin M180 | 15 | 15 | 15 |
| BIPHASIC INDUCING AGENT | | | |
| OF INSTANT INVENTION | | | |
| HI MAZE 260 (National Starch) | 5 | | |
| N-Oil ® or N-Lite ® (National | | Up to 5% | |
| Starch) | | | |
| BIPHASIC INDUCING AGENTS | | | |
| OF PRIOR ART | | | |
| ADJUNCT INGREDIENTS | | | |
| Perfume, colorant, preservatives | 1.5 | 1.5 | 1.5 |
| WATER | To 100% | To 100% | To 100% |
| Number of Aqueous Phases | 2 | 2 | 1 |
| After sitting w/o stirring/shaking | | | |
| Top Layer, cm | 3.5 | 5 | |
| Bottom Layer, cm (Turbid) | 3 | 1 | |
| Appearance of Shaken Sample | Turbid | — | |

### Example 9-10 and Comparative 8

Examples 9-10 illustrate the ability of other intermediate ethoxylate fatty esters to induce the formation of biphasic liquid cleanser when incorporated into a single aqueous phase reference composition, C8. The compositions were prepared by the methods described above. The compositions and results are set forth in Table 5 below.

The results in Table 5 demonstrate that both PEG-36 soya glyceride and PEG-80 sorbitan monolaurate, induce the formation of a biphasic liquid when used in combination with electrolyte and polydextrose.

TABLE 5
Compositions and results for Examples 9-10 and Comparative 8

| | | | C8 |
|---|---|---|---|
| | | | Ref for |
| INGREDIENTS | Ex 9 | Ex 10 | Ex 9-Ex 10 |
| SURFACTANT | Wt % | | |
| Ammonium Laureth sulfate | 4.6 | 4.6 | 4.6 |
| (1/2 EO) | | | |
| Ammonium Lauryl sulfate | 6.1 | 6.1 | 6.1 |
| Cocomonoethanolamide | 1.0 | 1.0 | 1.0 |
| PEG-5 | 0.5 | 0.5 | 0.5 |
| cocomonoethanolamide | | | |
| Alpha-step BSS-45^{a} | 2 | 2 | 2 |
| MgSO₄ | 3 | 3 | 3 |
| Maltrin M180 | 15 | 15 | 15 |
| BIPHASIC INDUCING AGENT | | | |
| OF INSTANT INVENTION | | | |
| PEG-36 Soya glyceride | 3 | — | |
| (Emulsogn EL 360 from | | | |
| Clariant) | | | |
| PEG-80 sorbitan monolaurate | — | 3 | |
| (Alkamuls PSML 80/72 from | | | |
| Rhodia) | | | |
| BIPHASIC INDUCING | | | |
| AGENTS OF PRIOR ART | | | |
| ADJUNCT INGREDIENTS | | | |
| Perfume, colorant, | 1.5 | 1.5 | 1.5 |
| preservatives | | | |
| WATER | To 100% | To 100% | To 100% |
| Number of Aqueous Phases | 2 | 2 | 1 |
| after sitting w/o stirring/shaking | | | |
| Top Layer, cm | 6 | 5 | |
| Bottom Layer, cm | 1.5 | 3 | |
| Appearance of Shaken | Turbid | Turbid | N/A |
| Sample | | | |

Example 11-15 in Table 6 further illustrate compositions of the invention

TABLE 6
Compositions and results for Examples 11-15

| INGREDIENTS | Ex 11 | Ex 12 | Ex 13 | Ex 14 | Ex 15 |
|---|---|---|---|---|---|
| SURFACTANT | Wt % | | | | |
| Sodium Laureth Sulfate | | 10 | 10 | 10 | 10 |
| Cocoamidopropylbetaine | | 3 | 3 | 3 | 3 |
| Ammonium Laureth sulfate | 4.6 | | | | |
| (1/2 EO) | | | | | |
| Ammonium Lauryl sulfate | 6.1 | | | | |
| Cocomonoethanolamide | 1.0 | | | | |
| PEG-5 | 0.5 | | | | |
| cocomonoethanolamide | | | | | |
| Alpha-step BSS-45^{a} | 2 | | | | |
| MgSO₄ | 3 | 5 | 3 | 3 | 4 |
| Maltrin M180 | | 10 | | 10 | 15 |
| BIPHASIC INDUCING | | | | | |
| AGENT OF INSTANT | | | | | |
| INVENTION | | | | | |
| Pullulan | 1 | | 3 | | |
| Gum Arabic | | 10 | | 5 | |
| HI MAIZE 260 | 15 | | | 5 | |
| Waxy maize (partially | | 2 | | | 3 |
| oxidized) | | | | | |
| PEG 50 Sorbitan laurate | 2 | | | | 1 |
| Laureth 50 | | 3 | | | 1 |
| PEG 30 Castor oil | | | | | 2 |
| ADJUNCT INGREDIENTS | | | | | |
| Perfume, colorant, | 1.5 | 1.5 | 1.5 | 1.5 | 1.5 |
| preservatives | | | | | |
| WATER | To 100% | To 100% | To 100% | To 100% | To 100% |

1. A liquid cleansing composition comprising:
a) about 5% to about 75% by wt. of a surfactant selected from the group consisting of anionic surfactants, nonionic surfactants, amphoteric/zwitterionic surfactants, cationic surfactants and mixtures thereof;
b) a Biphasic Inducing Agent selected from the group consisting of high-gel-point polysaccharides having a molecular weight greater than about 10,000 Daltons; intermediate ethoxylate fatty esters or fatty ethers; and mixtures thereof;
c) from 0 to about 10% of a salt;
d) from 0 to about 35% of a polydextrose;
wherein the amount of biphasic inducing agent is sufficient to produce at least two visibly separated aqueous based layers when the compositions are left undisturbed without stirring or shaking or other agitation.

2. A composition according to claim 1 comprising 6% to 40% surfactant.

3. A composition according to claim 1 wherein the molecular weight of the high-gel-point polysaccharide is at least about 50,000 Daltons.

4. A composition according to claim 1 wherein the high-gel-point polysaccharide is a pullulan or a high amylopectan starch or a depolymerized starch or an exudate gum or mixtures thereof.

5. A composition according to claim 4 wherein the pullulan has a molecular weight of at least 100,000, the high amylopectin starch is waxy maize, the depolymerized starch is an acid or enzyme converted tapioca, corn or rice starch and the exudate gum is gum arabic.

6. A composition according to claim 1 wherein the intermediate ethoxylate fatty ether and fatty ester has a degree of ethoxylation from about 20 to about 100.

7. A composition according to claim 6 wherein intermediate ethoxylate fatty ether is a C₁₂ to C₁₈ fatty alcohol ethoxylate and the fatty ester ethoxylate is a C₁₂ to C₂₂ fatty glyceride ethoxylate.

8. A composition according to claim 1 wherein the amount of biphasic inducing agent is at least 0.2% based on the total weight of the composition.

9. A composition according to claim 1 comprising from about 5% to about 20% surfactant and from about 0.2% to about 10% of the phase inducing agent.

10. A composition according to claim 1 comprising from about 5% to about 20% surfactant and from about 0.2% to about 10% of the Biphasic Inducing Agent.

11. A liquid cleansing composition comprising:
a) about 5% to 75% by wt. of a surfactant selected from the group consisting of anionic surfactants, nonionic surfactants, amphoteric/zwitterionic surfactants, cationic surfactants and mixtures thereof;
b) a Biphasic Inducing Agent selected from the group consisting of a high-gel-point polysaccharide having a molecular weight greater than about 10,000 Daltons; an intermediate ethoxylate fatty ester or fatty ether; and mixtures thereof;
c) from about 2% to about 10% a salt;
d) from about 5% to about 35% of a polydextrose;
e) balance water and minors; and
wherein the amount of biphasic inducing agent is sufficient to produce at least two visibly separated aqueous based layers when the compositions are left undisturbed without stirring or shaking or other agitation.

12. A composition according to claim 9 comprising from about 5% to about 20% surfactant and from about 0.2% to about 10% of the biphasic inducing agent.
